(19) United States
(12) Patent Application Publication
(10) Pub. No.: US 2010/0022753 A1
(43) Pub. Date: Jan. 28, 2010

(54) METHOD FOR MODIFYING THIOL GROUP

(75) Inventors: Yu-Wei Wu, Taipei (TW); Yu-Hui Tsai, Chiayi County (TW)

Correspondence Address: PAI PATENT & TRADEMARK LAW FIRM, 1001 FOURTH AVENUE, SUITE 3200, SEATTLE, WA, 98154, US

(73) Assignee: TAIPEI MEDICAL UNIVERSITY, Taipei (TW)

(21) Appl. No.: 12/179,584

(22) Filed: Jul. 25, 2008

(51) Int. Cl. C07K 1/00; C07H 21/00; C07K 2/00

(52) U.S. Cl. 530/362; 536/23.1; 530/300

(57) ABSTRACT

A method for modifying a thiol group is provided. The method includes: providing a first material containing at least one sulfhydryl group; providing a second material containing at least one thiourea group; and reacting the first material with the second material in the presence of copper (II) ions to quickly form a disulfide bond between the sulfhydryl group and the thiourea group, wherein the disulfide bond can be easily reduced.

### BACKGROUND OF THE INVENTION

1. Field of the Invention

The present invention relates to modification of a thiol group, and in particular relates to forming a reversible disulfide bond between a sulfhydryl group and a thiourea group in the presence of copper (II) ions.

2. Description of the Related Art

Thiourea (Tu) and its derivatives are widely used in the chemical, agricultural, metallurgical, and pharmaceutical industries [IPCS (2003) Thiourea, in CICADs 49 (2003 IPCS, Ed.) WHO, Geneva]. Exposure of humans and animals to Tu-containing compounds, which is considered an environmental risk factor, may cause various illnesses such as thyroid hyperplasia, pulmonary edema, pleural effusion, tumors, etc (Environ. Health Perspect. 85, 43-50; Toxicology 197, 81-91). Physiologically, Tu is absorbed from the gastrointestinal tract in humans and animals, and is mostly excreted unchanged in urine (J. Biol. Chem. 183, 215-221). However, in rat experiments, accumulation of radiolabeled Tu have been found mainly in the thyroid tissue, and to a lesser degree in the kidney, blood cells, lung, as well as liver (J. Biol. Chem. 183, 215-221; Drug Metab Dispos. 2, 521-525). Studies have shown that the metabolism of Tu in the thyroid gland and liver is quite different. In the thyroid gland, Tu is first oxidized by the thyroid peroxidase to form dithioformamidine (Tu-S—S-Tu), which is later decomposed to cyanamide, sulfur, and Tu. Both Tu and cyanamide are inhibitors of the thyroid peroxidase and may eventually lead to a dysfunctional thyroid gland (Endocrinology 104, 919-924). In the liver, Tu is metabolized by microsomal flavin-containing monooxygenase (FMO)-catalyzed S-oxygenation, to formamidine sulfenate and formamidine sulfinate (Biochem. Soc. Trans. 6, 94-96). In addition, radiolabeled Tu has also been found to covalently bind to the thyroid gland and lung proteins non-enzymatically (J. Biol. Chem. 236, 1689-1692; Toxicol. Lett. 52, 1-5). The authors speculated that the thiol-disulfide exchange between the thiol of Tu with the intra-molecular disulfide bridge of proteins, lead to the formation of protein-S—S-Tu complex, thus binding Tu to the thyroid proteins. Subsequently, the complex may be hydrolyzed to protein-S—SH and urea.

Tu is known to interact with sulfhydryl-containing compounds such as cysteine or glutathione in vitro (Biochem. J. 27, 1181-1188; J. Biol. Chem. 120, 297-313.). The oxidation of cysteine is catalyzed by Tu in the presence of hydrogen peroxide in acid solution. The formation of the Cys-S—S-Tu complex is found in the reaction of cysteine with equimolar or excess amounts of dithioformamidine (Tu-S—S-Tu). Cys-S—S-Tu is also produced by reacting cystine with Tu. Finally, Cys-S—S-Tu may be hydrolyzed spontaneously. However, the formation of Cys-S—S-Tu is not only substantially time-consuming (about few hours to few days) but also only occurs when the concentration of Tu-S—S-Tu excesses that of Cys-SH. Alternatively, Cys-S—S-Tu may also be produced by reacting cystine with Tu, which is a thiol-disulfide exchange chemical reaction. Similarly, this thiol-disulfide exchange reaction is also time-consuming. Thus, the production of Cys-S—S-Tu is poor.

Further, in order to label a marker to a thiol-material, some reagents such as iodoacetic acid, 5-I-Aedans, N-ethylmaleimide, and 3-bromoprohylamine are used for modifying thiol group. However, these agents will form an irreversible covalent bond with a sulfhydryl group, so that the labeled marker cannot be removed and reused.

Accordingly, what is needed in the art is a method for modifying a thiol group to form a reversible disulfide bond while at the same time providing the advantages of rapid reaction time and cost efficiency.

### BRIEF SUMMARY OF THE INVENTION

The present invention provides a method for modifying a thiol group, comprising: providing a first material containing at least one sulfhydryl group; providing a second material containing at least one thiourea group; and reacting the first material with the second material in the presence of copper (II) ion to form a disulfide bond between the first material and the second material.

The present invention provides a method for reversible labeling, comprising: providing a substance containing at least one sulfhydryl or thiourea group; providing a tag containing at least one thiourea group or sulfhydryl, respctively; and labeling the tag to the substance in the presence of copper (II) ions via at least one disulfide bond, wherein one of either the substance or the tag contains the sulfhydryl group, and the other contains the thiourea group

The present invention further provides a kit comprising a marker containing at least one sulfhydryl group or thiourea group; and a Cu (II) ion solution.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIGS. 1A-1B show the analysis of copper-induced binding of the present invention;

FIGS. 2A-2C show dose-dependence of HSA, FTC and Cu (II) ion on albumin thioureation.

FIG. 3A shows urea-, SDS, and DTT-treatment damaging the HSA-FTC complexes;

FIG. 3B shows that FTC does not interact with cysteinylated (Cys-HA1, Cys-HA2) or alkylated albumin (Alk-HA);

FIG. 3C shows a copper-induced binding method of the present invention;

FIG. 4 shows only Cu(II) inducing the thioureation reaction;

FIG. 5A shows that copper-induced HSA-FTC complex formation is greatly influenced by pH of the reaction solution;

FIG. 5B shows that the relative initial reaction rate constants of the reactions is influenced by various pHs;

FIG. 5C shows in the absence of albumin, the interaction between the FTC and Cu(II) is influenced by pH;

FIG. 6A shows NCP enhancing the formation of HSA-FTC complex;

FIG. 6B shows NCP enhancing the formation of Cys-S—S-FTC complex

FIG. 7A shows that the HSA-FTC complex is stable at 25° C., pH 7.0 within an hour;

FIG. 7B shows that the HSA-FTC complex is stable at pH 6.0-8.0 at 25° C. but degraded significantly in more acidic or basic conditions;

FIG. 7C shows that Cu(I) is capable of reducing the Alb-FTC complex;

FIG. 7D shows the thiol-containing agents causing the release of FTC from HSA-FTC complex; and

FIGS. 8A-8B show that FTC, FITC-insulin and FITC-KY15 are conjugated with HSA through a copper-dependent reaction of the present invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

In accordance with one aspect, the present invention provides a novel method for modifying a thiol group comprising: providing a first material containing at least one sulfhydryl group; providing a second material containing at least one thiourea group; and reacting the first material with the second material in the presence of copper (II) ions to form a disulfide bond between the first material and the second material.

The “sulfhydryl group” of the invention is a compound that contains the functional group composed of a sulfur atom and a hydrogen atom (—SH). This functional group is referred to as either a thiol group or a sulfhydryl group. More traditionally, thiols are often referred to as mercaptans. In the presence of a base, a thiolate anion is formed. The group and its corresponding anion are readily oxidized by reagents to give an organic disulfide (R—S—S—R).

The “thiourea group” of the invention is an organic compound of carbon, nitrogen, sulfur and hydrogen, with the formula CSN₂H₄ or (NH₂)₂CS. It is similar to urea, except that the oxygen atom is replaced by a sulfur atom. The properties of urea and thiourea differ significantly because of the relative electronegativities of sulfur and oxygen. Thiourea is a versatile reagent in organic synthesis. “Thioureas” refers to a broad class of compounds with the general structure (R¹R²N)(R³R⁴N)C═S.

The method of the present invention provides a novel reaction for forming a reversible disulfide bond, and the chemical reaction is shown in the Thiourea formula, R—SH+Cu(II)→Thiourea-S—S—R+Cu(I)+2H⁺. In the present invention, Cu(II) ions, are utilized as an oxidizing agent to catalyze the reaction of the thiourea and sulfhydryl groups.

The disulfide bond formed by the method of the present invention is a reversible covalent bond, and can be reduced or broken by reducing agents. Examples of the reducing agents include, but are not limited to, urea, sodium dodecyl sulfate (SDS), dithiothreitol (DTT), tri(carboxyethyl)phosphine (TCEP), dithioerythritol (DTE), 2-. Mercaptoethanol, calcium thioglycolate, or other disulfide cleavage reagents.

In the present invention, the disulfide bond can be speedily formed in the presence of Cu(II) ions as compared with a thiol-disulfide exchange. Cu(II) ions are used as an oxidizing agent to oxidize the sulfhydryl and thiourea groups resulting in the formation of the disulfide bond. Additionally, Cu(II) ions are reduced to form Cu(I) ions. Thus, the method of the present invention is distinct from the traditional thiol-disulfide exchange, and the formation of the disulfide bond in the present invention is significantly faster. In addition, the formation of the disulfide bond is positive dose-dependant upon copper content, so that the formation of the disulfide bond can be induced and increased by increasing the Cu(II) concentration. In one embodiment, the disulfide bond can be formed in less than 1 minute. In another embodiment, the formation time of the disulfide bond is about 5 to 30 minutes. The concentration of Cu(II) is not particularly limited, and one of ordinary skill in the art will select a suitable concentration according to the amount of the reactant. In the present invention, the formation of the disulfide bond is positively dose-dependent upon the concentration of copper (II) ion.

Further, The disulfide bond of the present invention can be broken by (a) thiol-reducing agents such as dithiothreitol; (b) high temperature (above 25° C.); (c) acidic pH (<6.0) or basic pH (>8.0); (d) the copper (I) ion at pH below 7.0.

The stability of the disulfide bond of the present invention may be changed dependant upon various temperatures and pH values. In one embodiment, the disulfide bond of the present invention appears stable at about a pH 5.0 to 9.0, preferably, about a pH 6.0 to 8.0, most preferably, about a pH 7.0. In another embodiment, the disulfide bond of the present invention is stable at 25° C., but may be unstable above 25° C., such as about 37 to 45° C. In another embodiment, the presence of thiol-containing agents such as DTT, NAC, cysteine, Tu, and ANTU may cause the reduction of disulfide bond.

Furthermore, the formation of the reversible disulfide bond is greatly enhanced and reaches its plateau if a Cu(I) chelator is added. The Cu(I) chelator includes, but are not limited to, NCP, bathocuproine, or bicinchoninic acid.

The materials containing the sulfhydryl or thiourea group can be any suitable materials. Examples of the materials include, but are not limited to a nucleic acid, a lipid, a fatty acid, a carbohydrate, a protein, a peptide, an amino acid, a chemical compound including natural and synthetic polymers, isotopes, fluorescent dyes, and etc, or derivative thereof. Additionally, the materials can be drugs, such as thiourea isoxyl, carbimazole, methimazole or propylthiouracil, 6-n-propylthiouracil (PTU), phenoxyethyl-thiourea-pyridines, etc. Also, the materials can be proteins, such as, fluorescence protein, antibody, or signal peptide. In one embodiment, the sulfhydryl or thiourea group can be inherent in the materials, such as the thiourea drugs. In another embodiment, the sulfhydryl or thiourea group can be attached to the materials using additional chemical or physical processes.

In accordance with another aspect, the present invention further provides a method for reversible labeling the sulfhydryl group with the thiourea group, comprising: providing a substance containing at least one sulfhydryl or thiourea group; providing a tag containing at least one thiourea or sulfhydryl group, respectively; and labeling the tag to the substrate in the presence of copper (II) ions by at least one disulfide bond, wherein one of either the substrate or the tag contains the sulfhydryl group, and the other contains the thiourea group.

As disclosed above, the disulfide bond can be formed or produced in the presence of Cu(II) ions to couple the substance with the tag. In order to enhance the formation of the disulfide bond, Cu(I) ion chelators can be added. In addition, the disulfide bond can be reduced by using reducing agents. In one embodiment, the tag can be a specific probe (ex. isotope-, fluorescence-, peptide-, etc.) labeled thiourea-containing molecule, and the substance can be a sulfhydryl-containing protein. After treatment with Cu(II) ions, the tag and the substance are cross-linked via disulfide bridge formation to form a specific “probe-labeled protein”. The coupling reaction can be enhanced by using Cu(I) chelating agents. In addition, the disulfide bond can be reduced by using reducing agents to release the probe from the target protein. In another embodiment, the substance can be a drug containing thiourea groups, and the tag can be a drug deliverer containing sulfhydryl groups. After treatment with the Cu(II) ions, the drug deliverer can be coupled with the drug to enhance delivery efficiency of the drug.

In accordance with yet another aspect, the present invention further provides a kit comprising a marker containing at least one sulfhydryl group or thiourea group and Cu (II) ions. The marker can be a nucleic acid, a lipid, a fatty acid, a carbohydrate, a protein, a peptide, an amino acid, or a chemical compound including synthetic polymers, isotopes, fluorescent dyes, and etc. The marker of the present invention can be used for easily binding or labeling a substance, and one of either the substrate or the marker contains the sulfhydryl group, and the other contains the thiourea group.

### EXAMPLES

### Example 1

### The Method for Modifying the Thiol Group in the Invention

Mercaptoalbumin (Alb-Cys³⁴-SH) was used in this Example. The preparation of the mercaptoalbumin was modified from a previous publication (Int. J. Pept. Protein Res. 24, 96-103). Briefly, 1 mM human serum albumin (HSA) (Swampscott, Mass., U.S.A.) in sodium phosphate buffer (0.1 M Na₂PO₄, pH 6.86, 0.3 M NaCl, and 1 mM EDTA) was treated with 5 mM DTT at 25° C. for 45 minutes. Then, the phosphate buffer of the specimen was changed to an HBS buffer (10 mM Hepes-pH 7.0, 0.15 M NaCl) by dialysis at 4° C. One mol of an albumin was confirmed to contain one mol of a free thiol by Ellman's method (Arch. Biochem. Biophys. 82, 70-77).

In Example 1, a high-throughput CFA method modified from a previous publication was used to analyze the binding capacity of albumin with fluorescein-5-isothiocyanate cadaverine (FTC), dansylcadaverine (DC), fluorescein-5-carboxamide cadaverine (FCC), or a.-naphthylthiourea (ANTU) (J. Biomol. Screen. 11, 836-843). Typically, 30 μl per well of protein samples (2 μM HSA, unless otherwise stated) dissolved in HBS buffer (10 mM Hepes-pH 7.0, 0.15 M NaCl) were added in a microplate at 4° C. After 30 μl of a freshly prepared reagent-A (100 mM Hepes-pH 7.0, 0.2 mM CuCl₂, and 6 μM FTC) was added into each well, a reaction was initiated by incubating the microplate in a 37° C. humidified incubator for 10 minutes, and terminated by adding 60 μl of a reagent-B (2 mM EDTA, 0.5 M Hepes-pH 8.0; 4° C.). A suspended MD-Charcoal (200 μl per well) was then added and incubated at room temperature for 5 minutes to remove all of the free fluorescent dyes. After precipitating the suspended MD-Charcoal by a magnet for 1 minute, the fluorescence intensity in each well was measured by a microplate reader (Plate Chameleon, Hidex Oy, Finland) with an excitation wavelength at 485 nm and emission wavelength at 535 nm (Ex485/Em535), gain35. The transfer of the supernatant to a new plate for the fluorescence counting was not necessary because the precipitated MD-Charcoal does not influence the fluorescence in supernatant. Background level of the fluorescence intensities were measured by replacing protein samples with HBS buffer. The binding capacity of the albumin for the fluorescent dye was expressed as a net or relative fluorescence (fluo.) intensity per reaction. All experiments were performed in triplicates for each datum point and the data were presented as means±SD.

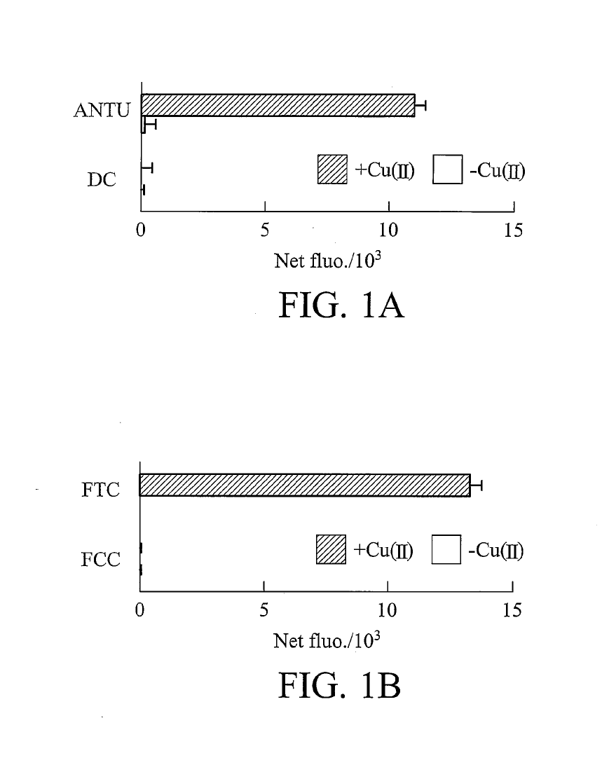

Referring to FIGS. 1A-1B, no dyes were bound to the Has, except for when the copper was added. In addition, the DC and FCC did not bond to the HSA, because not all of the DC and FCC had the thiourea functional group (Tu group). In comparison, ANTU and FTC having the similar Tu functional groups were bound to HSA. Thus, indicating that the Tu group interacted with the albumin upon copper induction.

### Example 2

### Effect of Dosage of HSA, FTC, and Cu (II) Ion

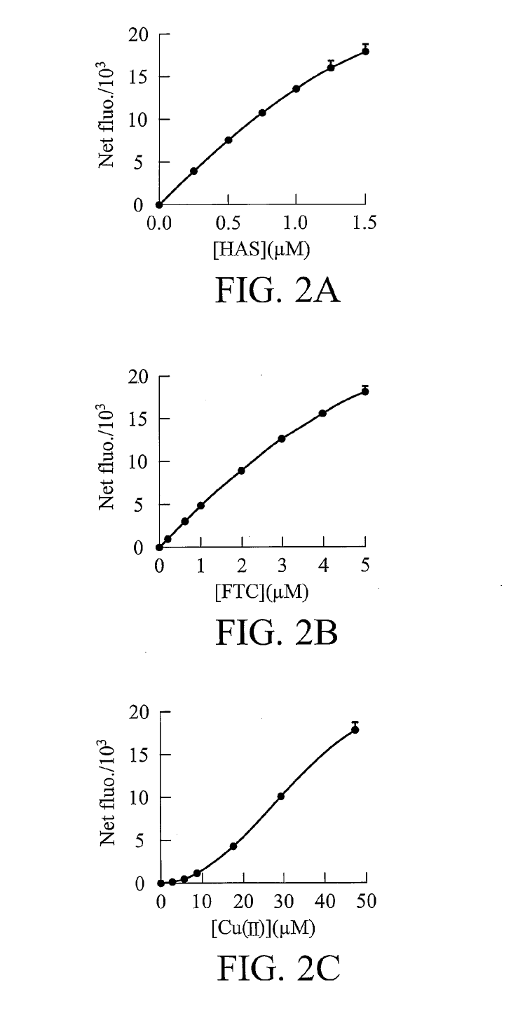

The same procedures carried out in Example 1 was repeated, except that HSA, FTC, and copper concentration was changed. The FTC was selected for the subsequent HSA binding analyses. Referring to FIGS. 2A-2C, the coupling reaction showed a positive dose-dependence on HSA, FTC, and copper contents.

### Example 3

### Disulfide Bridge Formation between the Tu Group and Cysteine

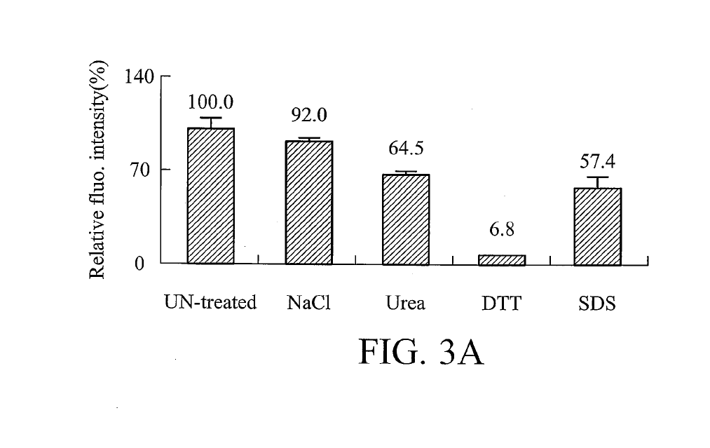

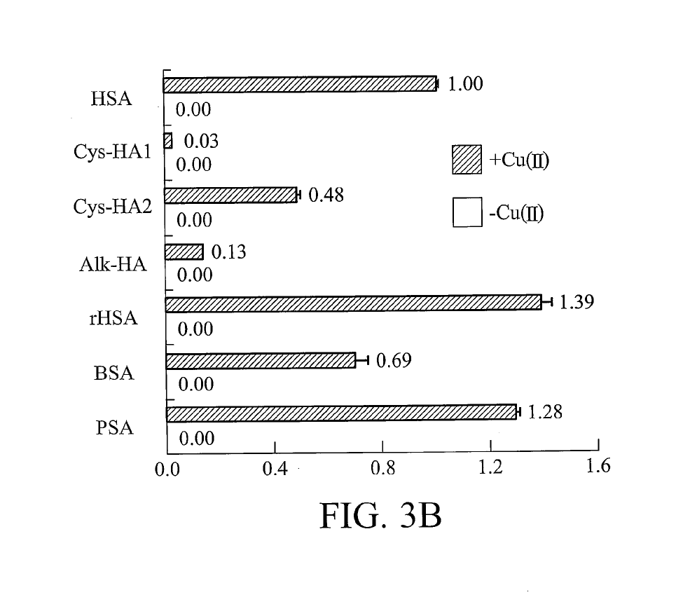

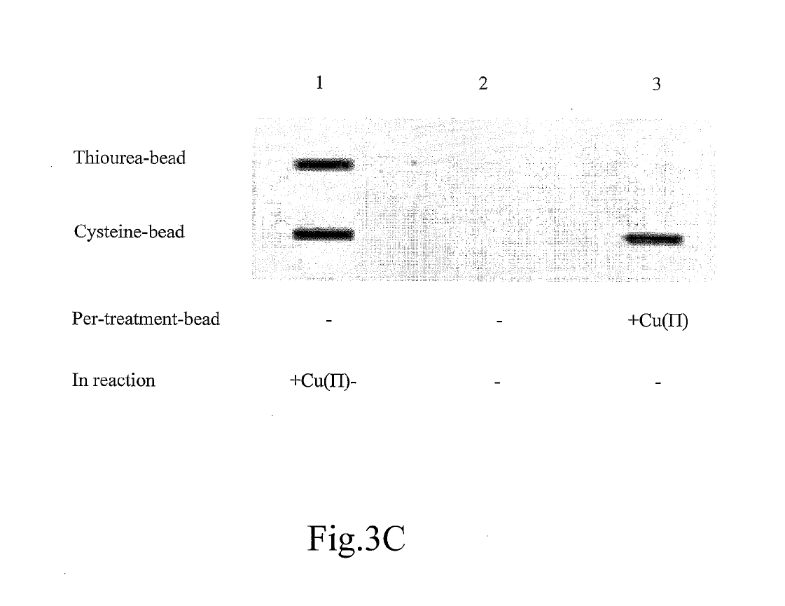

In order to identify the type of linkage between the HSA and FTC, the purified HSA-FTC complexes were treated with NaCl, urea, DTT, and SDS, respectively. Referring to FIG. 3A, urea- and SDS-treatment damaged up to 40% of the complexes, and the DTT-treatment almost eliminated the fluorescence signal of the protein fractions (>90%). However, a high concentration of salt (1 M NaCl) did not significantly influence the stability of HSA-FTC complexes. Thus, indicating that the linkage between HSA and FTC is a disulfide bond.

In addition, the Cys³⁴-modified albumins, Cys-HA1, Cys-HA2, or Alk-HA, was employed in the reaction. Alkylation of albumin (Alk-HA) was performed according to Glowacki et al. with modifications (J. Biol. Chem. 279, 10864-10871). Cysteinylation of albumin was performed as following: step 1, thiol-disulfide exchange—a reaction mixture containing 50 μM human mercaptoalbumin, 1 mM L-cystine, 0.1 M Hepes-pH 7.5, and 0.1 mM EDTA was incubated at 37° C. for 21 hours. The resulting buffer product (Cys-HA) was changed to HBS buffer by using a 30 K ultrafiltration unit (Amicon Ultra-15); step 2, alkylation—un-reacted —SH group of albumin was then alkylated by incubating 50 μM Cys-HA with 1 mM IAN, 0.1 M Hepes, pH 7.5, and 0.1 mM EDTA at 37° C. for 3 hours. The resulting buffer product (Cys-HA1) was changed to HBS buffer as described in step 1; step 3, reduction—Cys-HA1 (165 mM) was then treated with 5 mM DTT at room temperature for 45 minutes. The resulting buffer product (Cys-HA2) was changed again to HBS buffer as described in step 1. Free thiol content for Cys-HA1 and Cys-HA2 were calculated to be 0.08 and 0.50 (M/M), respectively. Referring to FIG. 3B, the FTC did not interact with the cysteinylated albumin (Cys-HA1, Cys-HA2) or alkylated albumin (Alk-HA). Thus, indicating that the binding capacity of the FTC with Cys-HA1, Cys-HA2, or Alk-HA, corresponded to the free thiol content, as compared to that with HSA.

Furthermore, Tu-bead was also used to confirm the participation of the Tu group of the FTC in the disulfide bridge formation, wherein the Cys-bead was used as the control. The Tu- and Cys-beads were prepared by coupling each ligand with an NHS-activated Sepharose 4 matrix. In detail, the Tu (0.1 M) or cysteine (0.1 M) dissolved in PBS buffer (137 mM NaCl, 2.7 mM KCl, 10 mM Na₂HPO₄, 2 mM KH₂PO₄, pH 7.4) was mixed with equal volume of the drained NHS-activated Sepharose 4 Fast Flow media at 25° C. for 2 hours with constant shaking. The resulting matrix was washed with a basic buffer (0.5 M Tris, pH 9.0, 0.5 M NaCl) and then with an acidic buffer (0.1 M acetate, 0.5 M NaCl, pH 4.0) for 3 cycles. Finally, the beads were washed with HBS buffer and stored at 4° C. The Cys-bead was treated with 10 mM DTT at 25° C. for 30 minutes before use. Alkylation of the Cys-bead (Alk-Cys-bead) was performed by incubating the bead with equal volume of 10 mM iodoacetamide at 37° C. for an hour with shaking. The bead was then washed with HBS buffer and stored at 4° C. The Tu- or Cys-beads with equal volume of 5 mM CuCl₂ at 25° C. for 15 minutes with constant shaking was mixed to form the Copper pre-treated Tu- or Cys-beads. After supplemented with 20 mM EDTA, the beads were then washed with HBS buffer and then stored at 4° C. till use.

Referring to FIG. 3C, in the presence of copper, the HSA was also found to form a disulfide bond with the Tu-bead. The same phenomenon was also observed for the Cys-bead. In the absence of copper, however, only the Cys-bead that was pre-treated with copper could bind with the HSA. The mechanism of the copper-induced cysteinylation appears different from that of the copper-dependent thioureation of the HSA.

### Example 4

### Effect of Metal Ions on the Thioureation Reaction

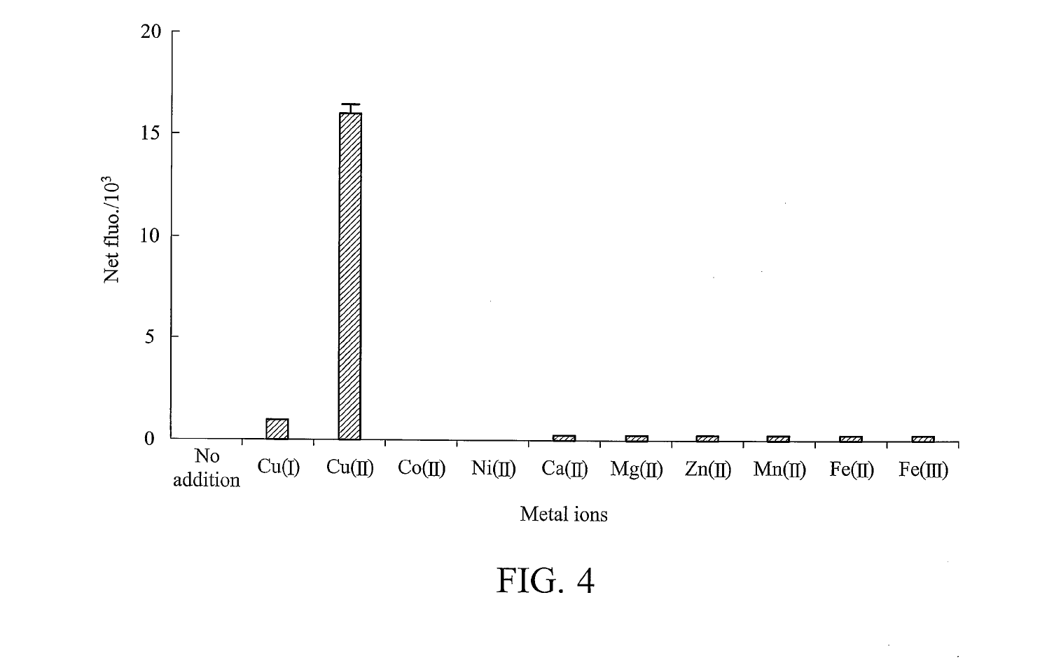

Various metal ions (0.1 mM in reaction) including CuCl, CuCl₂, CoCl₂, NiCl₂, CaCl₂, MgCl₂, ZnCl₂, MnCl₂, FeCl₂, and FeCl₃, were tested for Alb-FTC complex formation by using the CFA method. Referring to FIG. 4, only Cu (II) induced the thioureation reaction among the various metal ions; and the small amplitude of Cu (I)-induced reaction may have been the result of the oxidation of Cu(I) to Cu(II) during material preparation.

### Example 5

### Effect of pH on the Thioureation Reaction

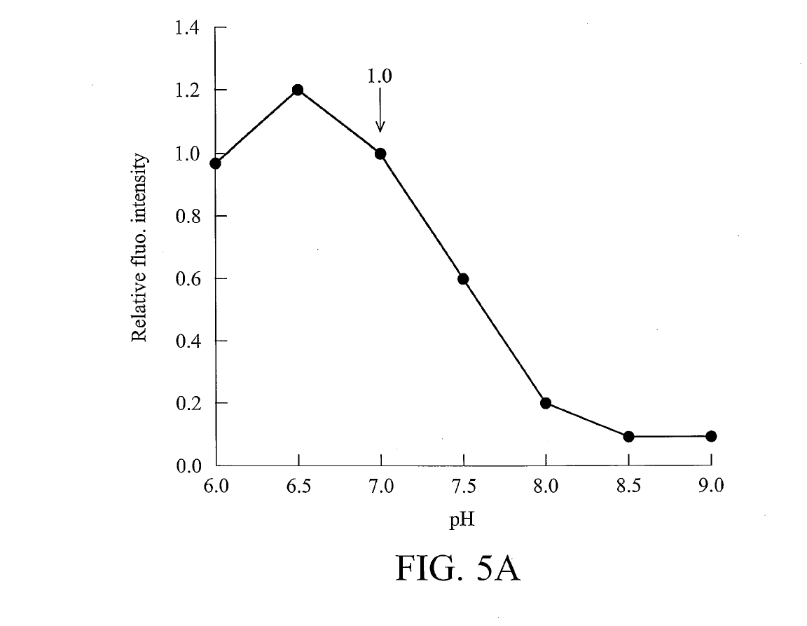

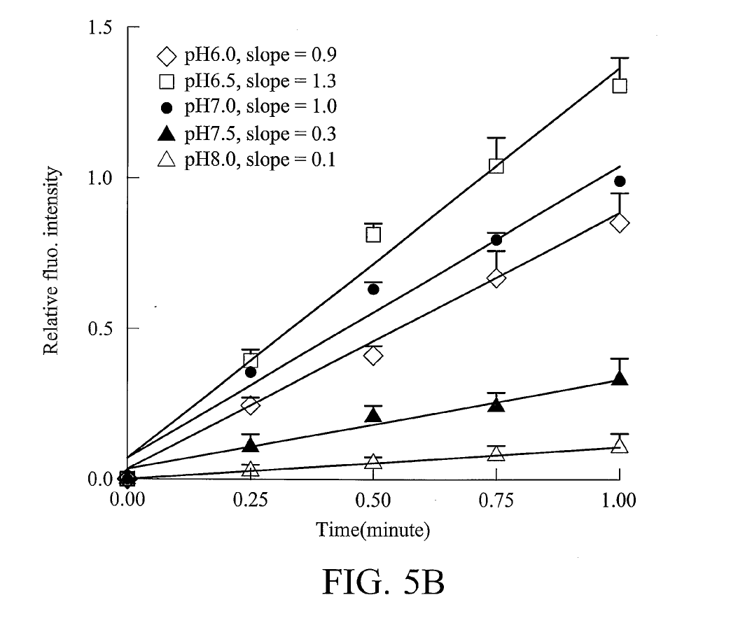

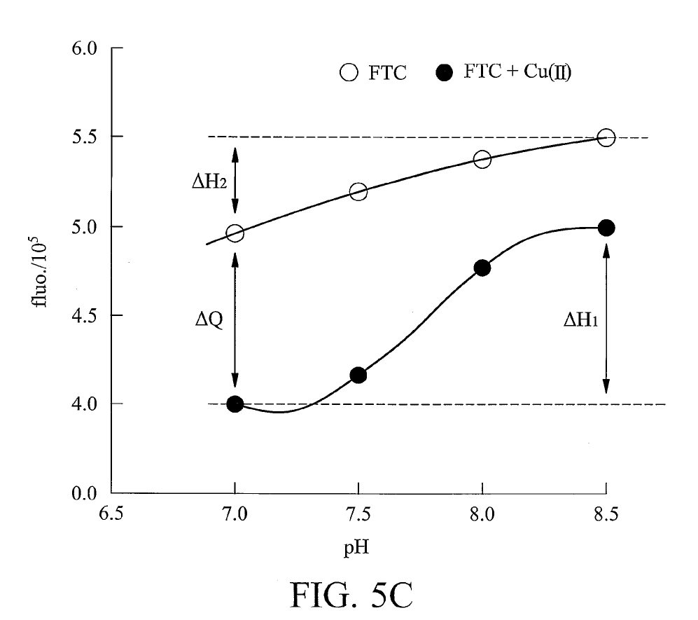

Reaction mixtures of protein and Reagents as described in Example 1 were incubated at various pHs at 37° C. for 10 minutes to evaluate the influence of pH on the formation of the HSA-FTC complex. Referring to FIG. 5A, the copper-induced HSA-FTC complex formation was greatly influenced by the pH of the reaction solution. The optimal pH for the reaction was at pH 6.5. For reaction above pH 6.5, the reaction was gradually reduced when increasing the pH, and was completely abolished at pH above 8.0. Referring to FIG. 5B, the relative initial reaction rate constants of the reactions at various pHs were also calculated, and the kinetic data did support the observations shown in FIG. 5A. Referring to FIG. 5C, in the absence of albumin, the interaction between the FTC and Cu(II) was also influenced by pH. Upon Cu(II) supplement, the fluorescence intensity of the FTC is severely quenched (ΔQ). However, the fluorescence of the FTC-Cu(II) mixture was greatly enhanced (ΔH₁) when the pH increased above 7.5, as compared to that observed in the absence of copper (ΔH₂). The increased fluorescence intensity (ΔH₁) at a high pH was speculated to be a result of the dissociation of the FTC from the Cu(II) ions.

### Example 6

### Effect of Cu (I) Chelator on the Thioureation Reaction

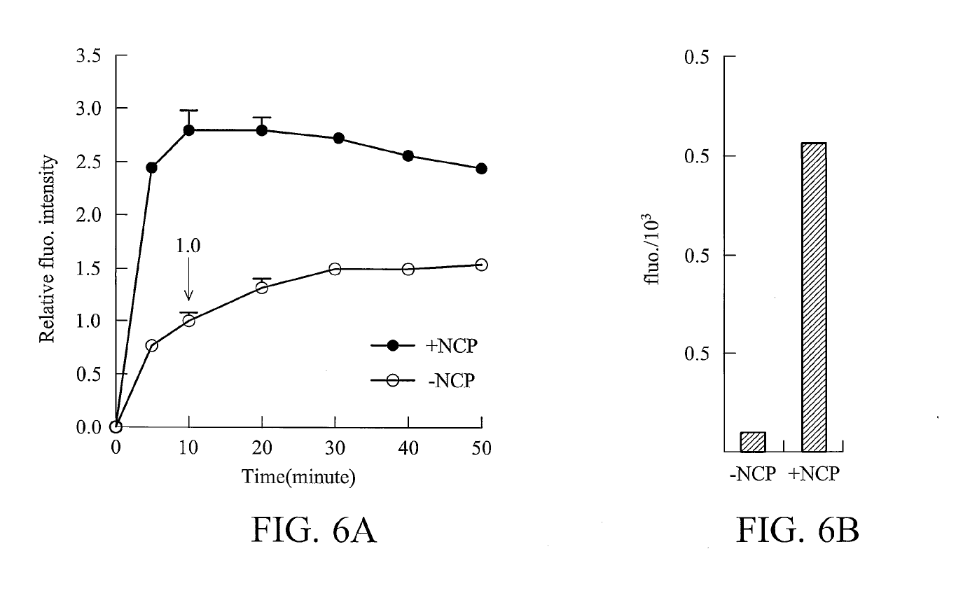

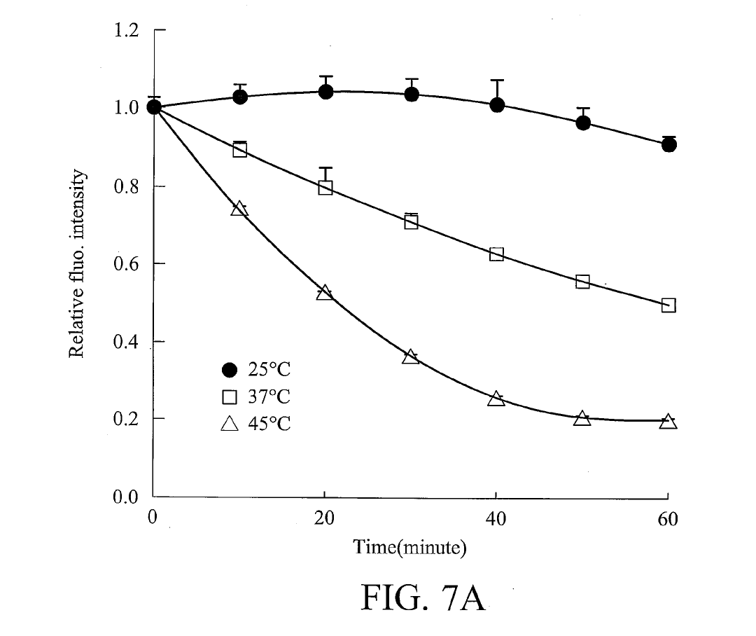

The reaction mixtures as described in Example 1 were incubated at 37° C. in the presence or absence of the NCP for indicated time-intervals. The NCP as the specific Cu(I) chelator was used to confirm the formation of the Cu(I). Referring to FIG. 6A, in the presence of the NCP, the formation of the HSA-FTC was greatly enhanced and reached its plateau within 10 minutes as compared to that observed in the absence of NCP. Similarly, the cross-linking of the FTC with cysteine (which was evaluated by Cys-bead) was also elevated by the NCP treatment as shown in FIG. 6B. In addition, the supplement of the Cu(I) did decrease the Cu(II)-induced formation of the HSA-FTC complexes. The results indicated that the Cu(I) was a byproduct associated with the Cu(II)-induced thioureation of Cys³⁴ of albumin, and the released Cu(I) would back inhibit the thioureation.

### Example 7

### The Stability of the Thioureated Products

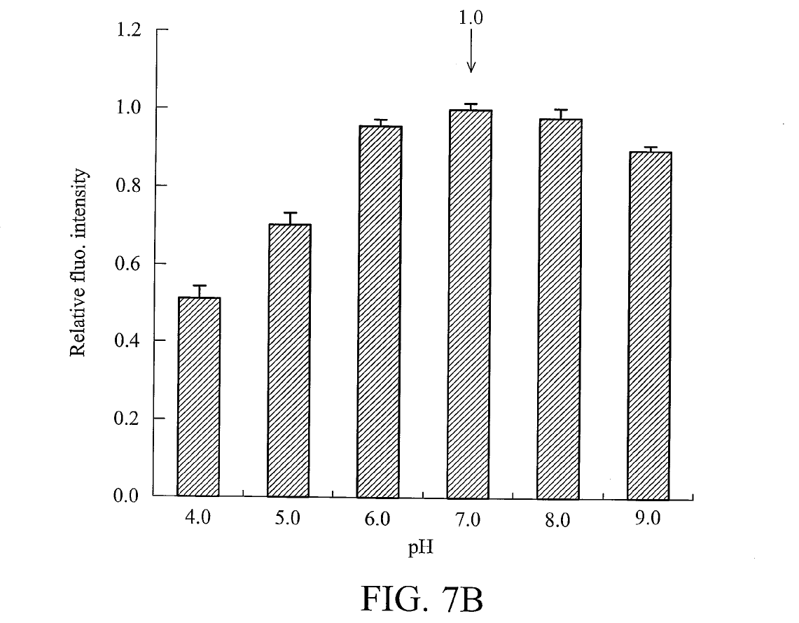

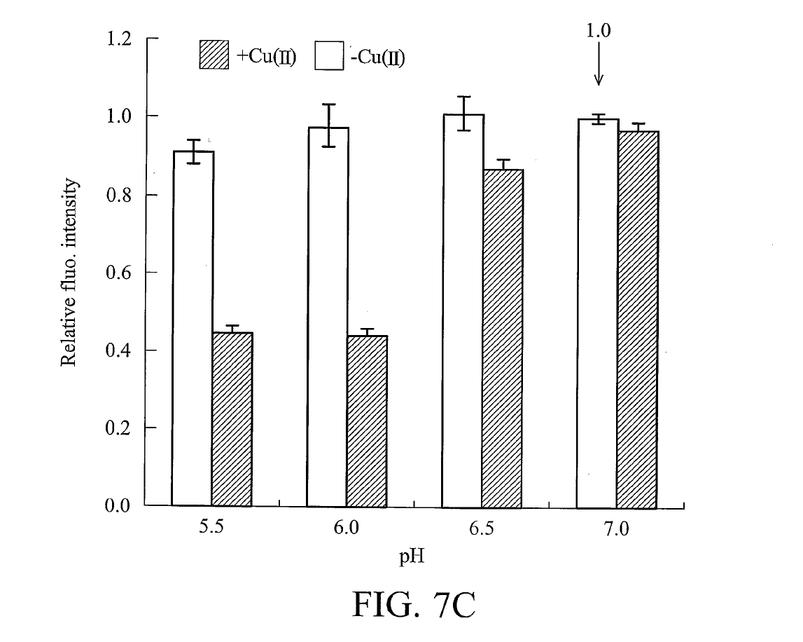

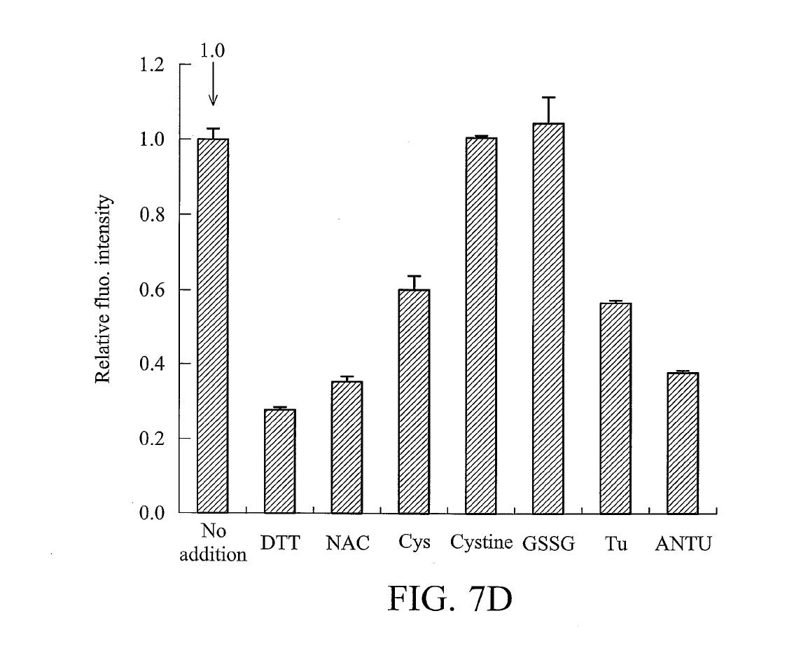

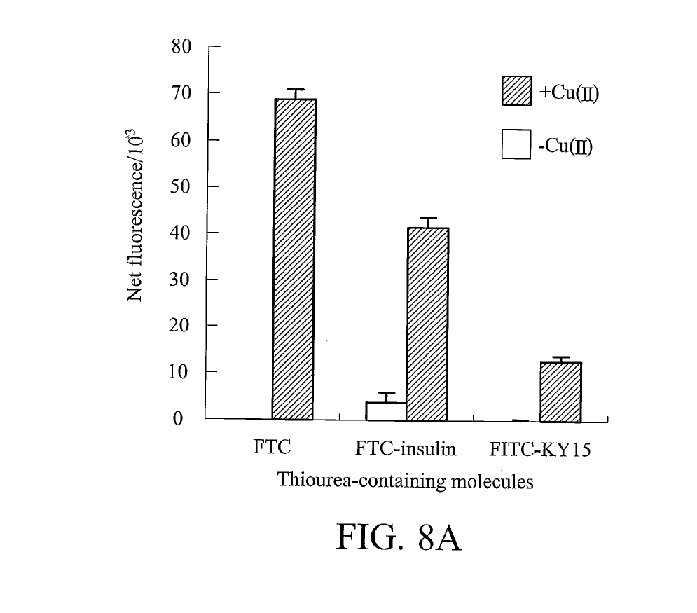

The HSA-FTC complex purified in Example 1 was used for a stability evaluation. The HSA-FTC complex was treated with various temperatures, pH values, and thiol-containing agents including DTT, NAC, cysteine, and Tu, ANTU. Referring to FIG. 7A, the HSA-FTC complex appeared stable at 25° C., pH 7.0 within an hour, but unstable at higher increased temperatures. Referring to FIG. 7B, the HSA-FTC complex was stable at pH 6.0-8.0 at 25° C. but degraded significantly in acidic or basic conditions. In acidic conditions, the Cu(I) was capable of reducing the HSA-FTC complex as shown in FIG. 7C. Further, referring to FIG. 7D, in the presence of the thiol-containing agents including DTT, NAC, cysteine, and Tu, ANTU caused the release of the FTC from the HSA-FTC complex. However, cystine and GSSG did not reduce the linkage of the HSA-FTC.

### Example 8

### Coupling of Peptide to a Protein Via Disulfide Bridge Formation

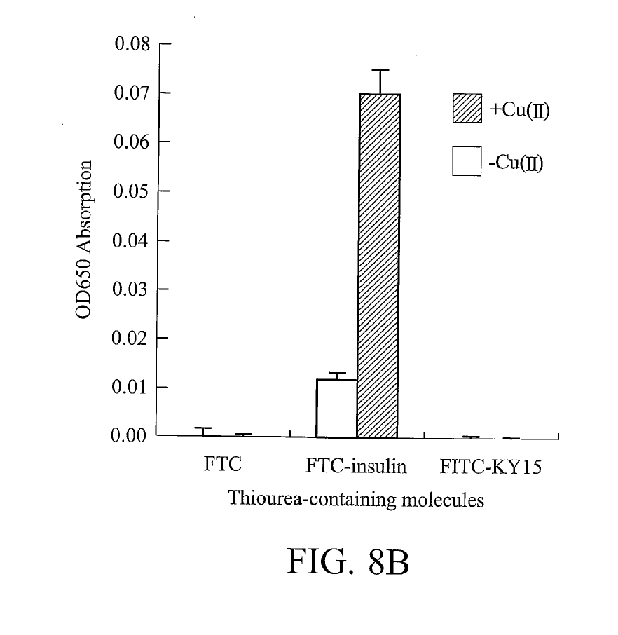

The same procedure carried out in Example 1 was repeated, wherein the insulin or peptide KY15 was labeled with/without FITC to form a “thiourea-containing peptide”. Using the CFA method, FTC, FITC-insulin and FITC-KY15 were found to conjugate with the HSA (a “sulfhydryl-containing protein”) through a copper-dependent reaction as shown in FIG. 8A.

In addition, in order to confirm that the FITC-labeled molecules were exactly coupled with the HSA, the resulting conjugated products remaining in the supernatants were coated in ELISA wells and then treated with an anti-insulin antibody. Referring to FIG. 8B, only FITC-insulin was detected. The results indicated that the FITC-labeled insulin conjugated with albumin.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for reversible modification of thiol groups, comprising:
providing a first material containing at least one sulfhydryl group;
providing a second material containing at least one thiourea group;
and reacting the sulfhydryl group of the first material with the thiourea group of the second material in the presence of copper (II) ion to form a disulfide bond between the first material and the second material.

2. The method as claimed in claim 1, wherein the formation of the disulfide bond is positively dose-dependent upon the concentration of copper (II) ion.

3. The method as claimed in claim 1, wherein the first material and the second material are reacted at a pH range from about 6.0 to about 8.0.

4. The method as claimed in claim 1, wherein Cu (I) ion is released at the step of reacting the first material and the second material.

5. The method as claimed in claim 1, addition of copper (I) chelator to the reaction enhances the coupling reaction between the first material and the second material.

6. The method as claimed in claim 1, wherein the disulfide bond is broken by thiol-reducing agents.

7. The method as claimed in claim 1, wherein the disulfide bond is broken at temperature above 25° C.

8. The method as claimed in claim 1, wherein the disulfide bond is broken by acidic pH (<6.0) or basic pH (>8.0).

9. The method as claimed in claim 1, wherein the disulfide bond is broken by the copper (I) ion at pH below 7.0.

10. The method as claimed in claim 1, wherein the first and/or the second material comprises a nucleic acid, a lipid, a fatty acid, a carbohydrate, a protein, a peptide, an amino acid, a chemical compound including natural and synthetic polymers, isotopes or fluorescent dyes, respectively.
